(19) United States
(12) Patent Application Publication
(10) Pub. No.: US 2013/0137919 A1
(43) Pub. Date: May 30, 2013

(54) MEDITATION ENHANCEMENT BOX HAVING MAGNETS AND BOADING BALL INSIDE

(71) Applicant: Eric Pepin, Portland, OR (US)

(72) Inventor: Eric Pepin, Portland, OR (US)

(21) Appl. No.: 13/306,958

(22) Filed: Nov. 29, 2011

(51) Int. Cl. A61M 21/00

(52) U.S. Cl. 600/28

(57) ABSTRACT

Meditation enhancement box is made of brass or copper, with magnets bonded to each corner and boading ball. The box can be a cube of about 5.08 cm. All magnets are pointed to center of box using a same polarity, and chime sound is produced during usage. Method for using box for meditation as follows: box rests in middle of palm in one hand, feeling weight of box and moving hand slowly clockwise and counterclockwise in circles, and vertical circles, initiating rhythmic breathing while moving hand; upon reaching relaxed state, index finger is placed on top of box and moving from middle towards edge of box in circles; turning over box to one adjacent side in a direction using free hand, followed by breathing in and out for 10 to 15 seconds while rotating the box in one adjacent side, and followed by rotating in another adjacent side, respectively.

### FIELD OF THE INVENTION

The present invention relates generally to a metal meditation enhancement box and in particular to a metal meditation enhancement box or cube made of copper or brass having a plurality of magnets and one Baoding ball inside.

### BACKGROUND OF THE INVENTION

Meditation and healing enhancement devices have been around for millennia. Some people have used copper and other metals for healing dating back for many centuries, and in many cultures, copper is known to be a strong transmitter of energy. Referring to U.S. Pat. No. 6,193,577, magnets in countries such as China, India, Egypt and Greece, have been used for healing for more than 4,000 years. Today, magnets are increasingly used and are widely-accepted to improve upon pain relief, healing, circulation and general health. It is known that magnetic wave therapy is said to fortify, balance and unblock the stagnant energy within human bodies.

Some one has previously invented a 3.5 inches cube-shaped metal meditation enhancement object for holding in the hands of the meditation participant during meditation and has named it “Metatron's Cube.” However, today, there has not been many known scientifically-proven meditation enhancement device that are able to provide therapeutic benefits such as, for example, increased relaxation, improved meditation, stress reduction, and other psychological, physiological or emotional benefits which are readily available to the public.

As a result, there is a need for an effective meditation enhancement device for improving meditation, relaxation and reducing stress, and perhaps achieving other benefits.

### SUMMARY OF THE INVENTION

An object of the invention is to provide a metal meditation enhancement box having a plurality of magnets and one Baoding ball inside, and the manufacturing method of the metal meditation enhancement box thereof.

Another object of the invention is to provide a meditation and breathing method in combination with using the aforementioned metal meditation enhancement box.

To achieve the foregoing objects, the metal meditation enhancement box is made of brass or copper. A plurality of magnets and one Baoding ball are placed inside the metal meditation enhancement box. A copper wire can be further bonded and conductively attached all around the metal meditation enhancement box. Meanwhile, another copper wire can also be bonded and conductively attached all around the Baoding ball which sits inside the meditation enhancement box, with one end of the copper wire connected to one magnet at one corner, and the other end of the copper wire connected to another magnet located in the farthest respective corner inside the metal meditation enhancement box.

Another object of the invention is to improving conductivity of the metal Baoding ball by soaking it in wine and then followed by drying.

Another object of the invention is to incorporating the magnets at each corner inside the meditation enhancement box, so that the respective same magnetic polarity of each of the magnets is pointed substantially toward the center of the copper meditation enhancement box, or alternatively, the same polarity of all of the magnets can be pointed not substantially to the center of the box, but that each pair of adjacent magnets have the same polarity pointed towards each other.

Another object of the invention is to constructing an empty copper box of a size of about 5.08 cm×5.08 cm×5.08 cm.

To achieve the foregoing objects, a manufacturing method for the metal meditation enhancement box is provided. The manufacturing method includes at least the following: fabricating of the copper or brass meditation enhancement box by metal casting two sections and a bottom plate, attaching 8 magnets, where one magnet is at each corner of the metal meditation enhancement box by either adhesive, welding, or other conventional bonding techniques, placing one Baoding ball in the center of the box, attaching one copper wire around the Baoding ball (this is an optional step), sealing the mediation box completely, and attaching one copper wire to the mediation cube by bonding the copper wire around in the middle region at 4 adjacent sides of the metal meditation cube (this is an optional step).

Another object of the invention is to provide a method for using the meditation enhancement box to obtain benefits or utility such as substantial relaxation, improved meditation quality or feelings of reduced “mind chatter”, and stress reduction. Such usage method of the metal meditation enhancement box may involve the turning over or rotating of the copper box on one adjacent face at a time so as to avoid hearing any chime rings made from inside the box through various manipulations by the hands of a meditation participant.

Another object of the invention is to provide a method for using the meditation enhancement box by incorporating various breathing and meditation techniques during the turning over and rotating of the copper box on one adjacent face at a time.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 shows an empty metal box for a metal meditation enhancement box according to an embodiment of the present invention;

FIG. 2 shows a plurality of individual parts which are assembled to form the empty metal box of FIG. 1;

FIG. 3 shows how the empty metal box is assembled together from the individual parts shown in FIG. 2;

FIG. 4 shows another perspective view of FIG. 3;

FIG. 5 shows how the metal meditation enhancement box is used by a meditation participant, wherein the metal meditation enhancement box is placed in the middle of the palm in one hand;

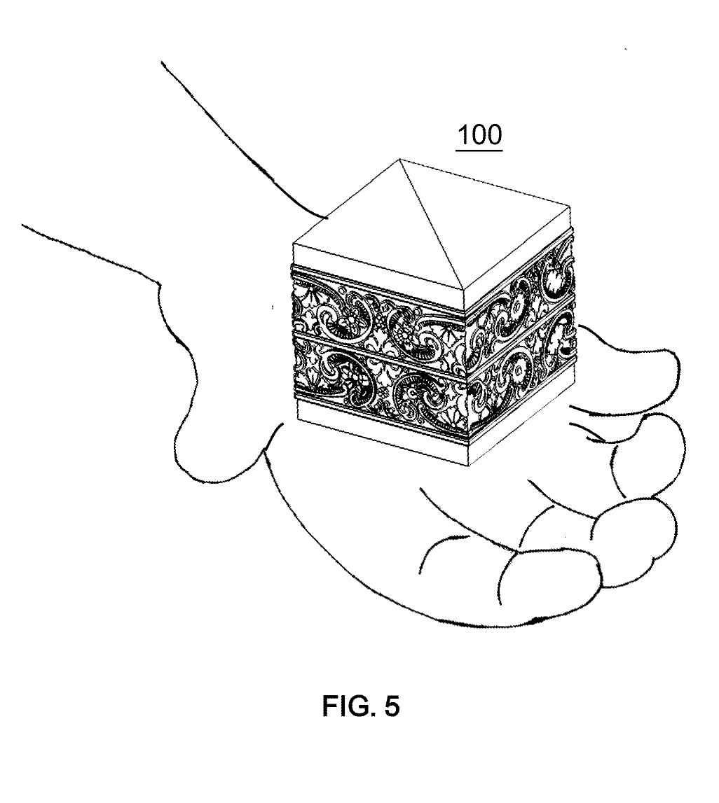

FIG. 6 shows a metal meditation enhancement box according to a first embodiment;

FIG. 7 shows a metal meditation enhancement box according to a second embodiment;

FIG. 8 shows a metal meditation enhancement box according to a third embodiment;

FIG. 9 is a flowchart showing a manufacturing method for fabricating the metal meditation enhancement box of the first, second, and third embodiments of present application; and

FIG. 10 is a flowchart showing an embodiment for a method for using the metal meditation enhancement box of the first, second, and third embodiments in a meditation session.

### DESCRIPTION OF THE PREFERRED EMBODIMENTS

While the present invention is susceptible of embodiments in various forms, there is shown in the drawings and will hereinafter be described a presently preferred embodiment with the understanding that the present disclosure is to be considered an exemplification of the invention and is not intended to limit the invention to the specific embodiment illustrated.

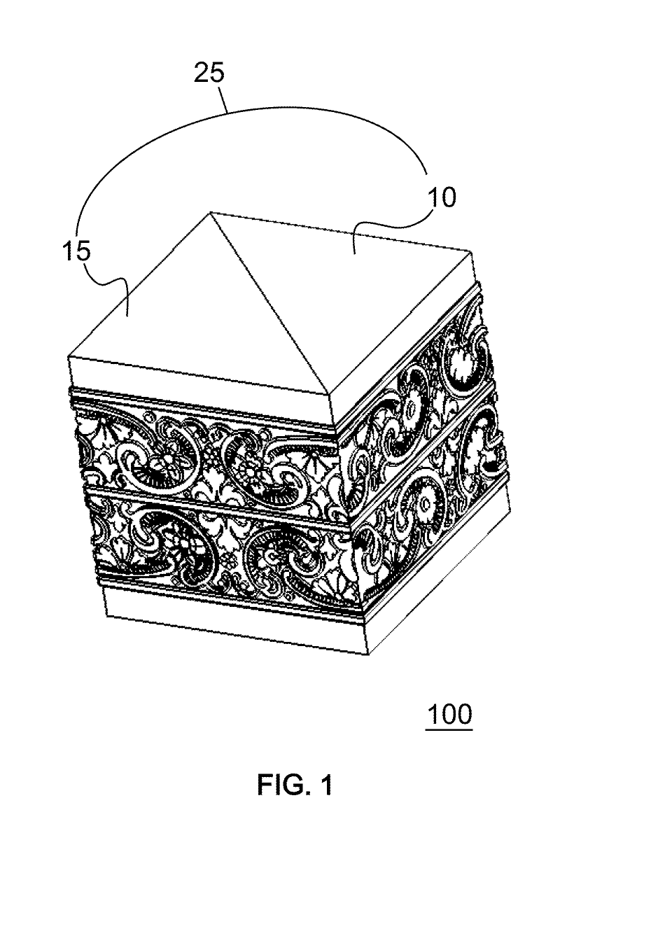

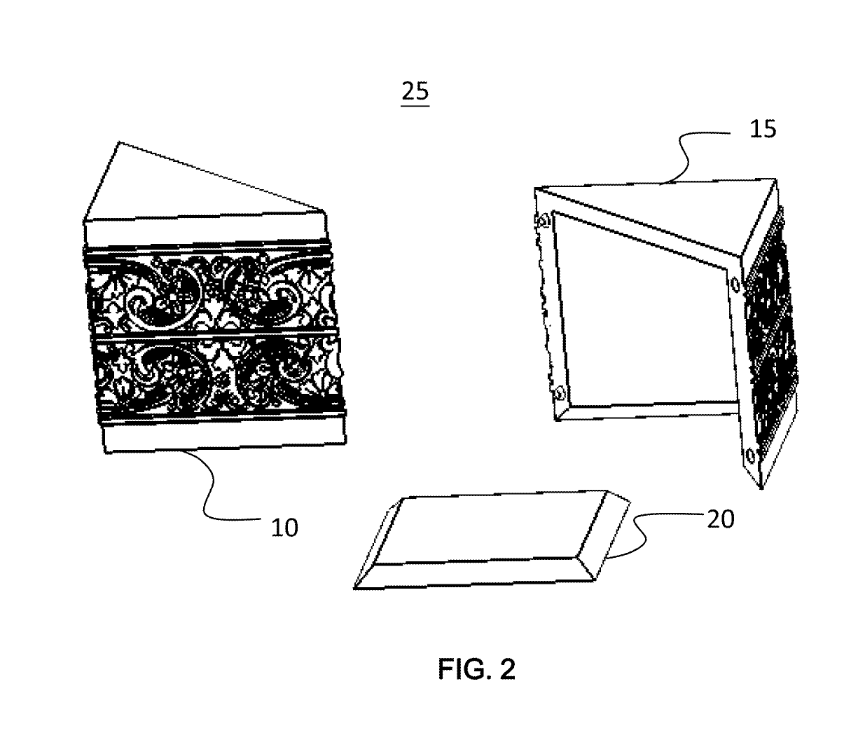

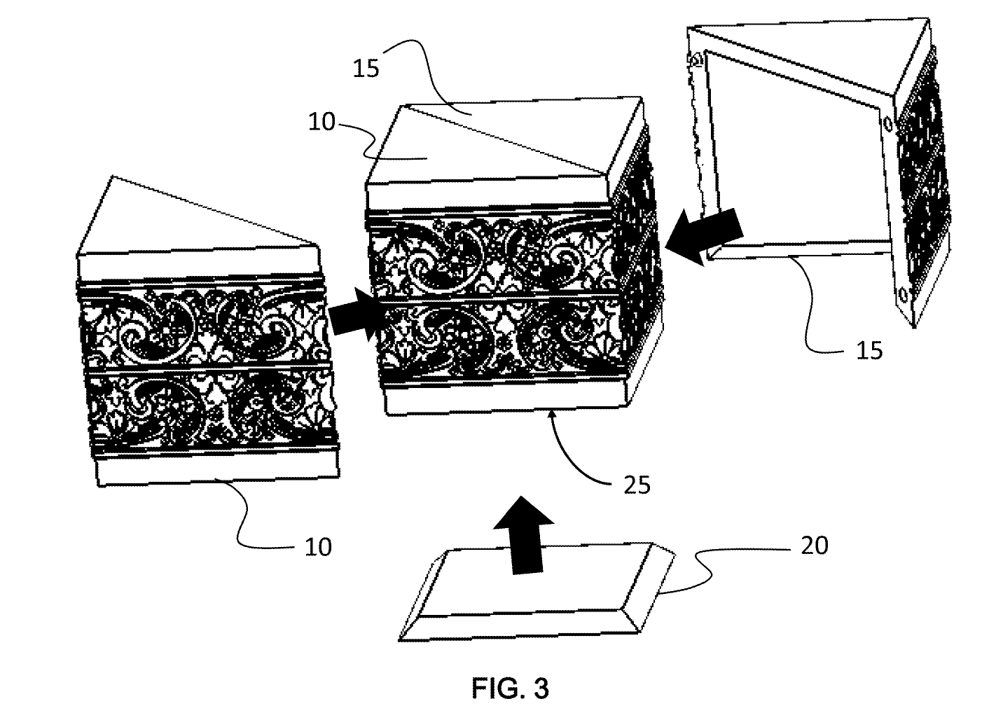

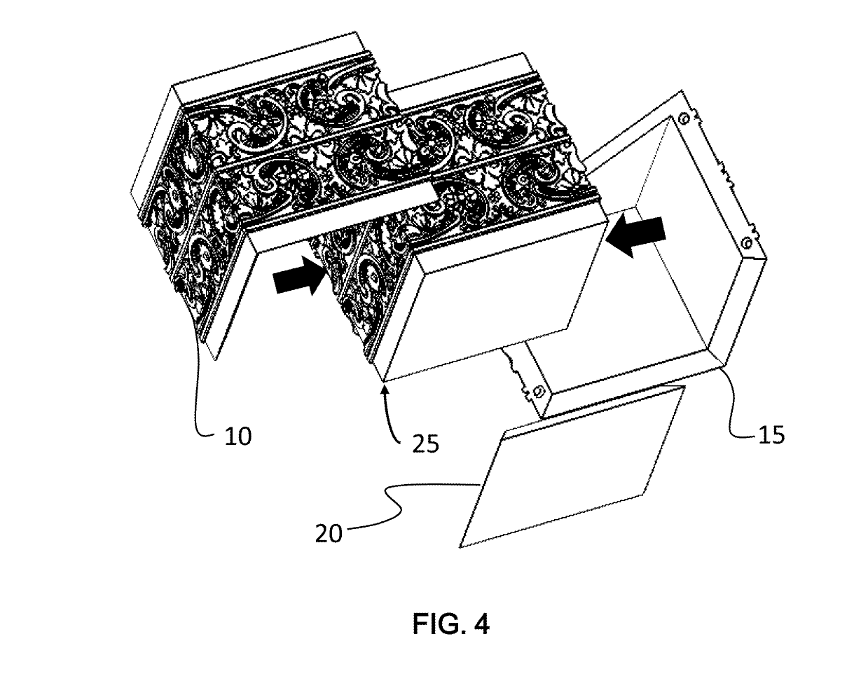

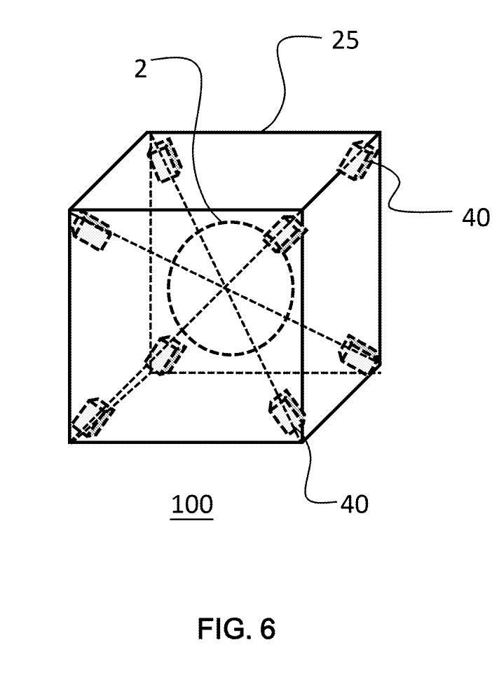

Please refer to FIGS. 1 and 6, a metal meditation enhancement box 100 made of brass or copper according to a first embodiment of the present invention is shown. The metal meditation enhancement box 100 includes an empty metal box 25 about 5.08 cm×5.08 cm×5.08 cm in size, which essentially is a cube shape. Referring also to FIGS. 2-4, the empty metal box 25 includes a first section 10, a second section 15, and a bottom plate 20. Referring to FIG. 6, a total of 8 magnets 40 made of neodymium (NdFeB) comprising of axial magnetization are bonded to each of the respective corners inside the empty metal box 25 via adhesive or epoxy glue, in which the same polarity of all of the magnets 40 are pointed substantially to the center of the empty metal box 25. The size of each magnet 40 is about 0.635 cm×0.635 cm (¼ inch×¼ inch). The shape of the magnet 40 can be for example cylindical, cubic, or bar, etc. . . . . In FIG. 2, the magnet 40 takes on the shape of a rectangular bar. One Baoding ball 2 about 33.5-35.5 mm in diameter is disposed inside the meditation enhancement box 100. The Boading ball 2 contains a chime inside to produce the chime sound during use of the metal meditation enhancement box 100 for meditation. A copper wire (not shown) can be further bonded and conductively attached all around the metal meditation enhancement box 100. In the following description herein, the metal meditation enhancement box 100, 200, 300 can also be referred to as the meditation enhancement box, or the box, for short.

Alternatively, conductivity of the metal Baoding ball 2 disposed in the metal meditation enhancement box 100 can be improved upon soaking it in wine and then followed by drying.

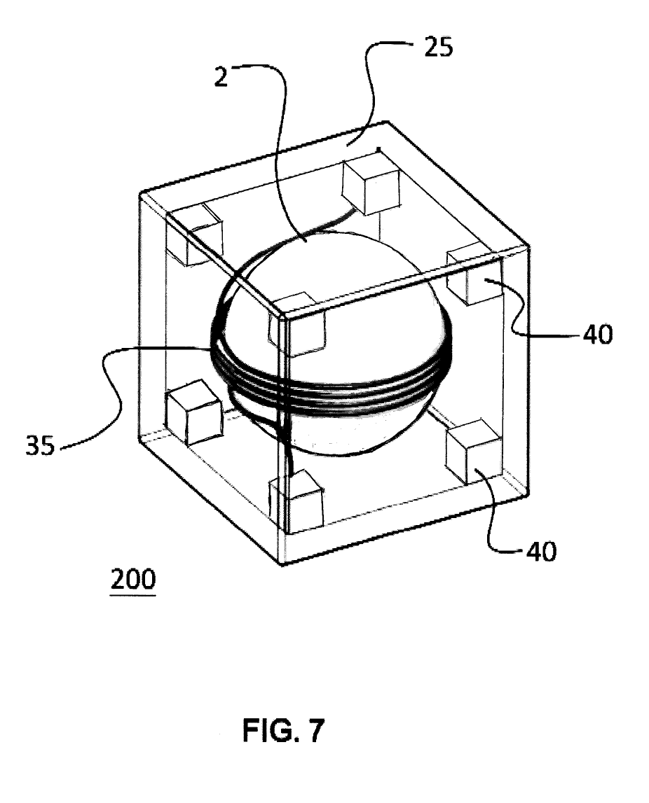

Referring to FIG. 7, in a second embodiment of the present invention, a metal meditation enhancement box 200 made of brass or copper is provided. The metal meditation enhancement box 200 includes an empty metal box about 44 mm×44 mm×44 mm in size, which essentially is a cube. A total of 8 magnets 40 made of neodymium are bonded to each of the respective corners inside the box via adhesive or epoxy glue, in which the same polarity of all of the magnets 40 are not pointed substantially to the center of the box, but wherein each pair of magnet 40 have the same polarity pointed towards one another (forming a total of 4 pairs of magnets). The size of each magnet 40 is 0.635 cm×0.635 cm. The shape of each magnet 40 is a cube. One Baoding ball 2 about 33.5-35.5 mm in diameter is disposed inside the metal meditation enhancement box 200. The Boading ball contains a chime inside to produce the chime sound or ring during usage of the metal meditation enhancement box 200 for meditation. A copper wire 35 is further bonded and conductively attached all around the Baoding ball 2 in the second embodiment, with one end of the copper wire 35 connected to one magnet 40, and the other end of the copper wire 35 connected to another magnet 40 located in the farthest respective corner inside the metal meditation enhancement box 200.

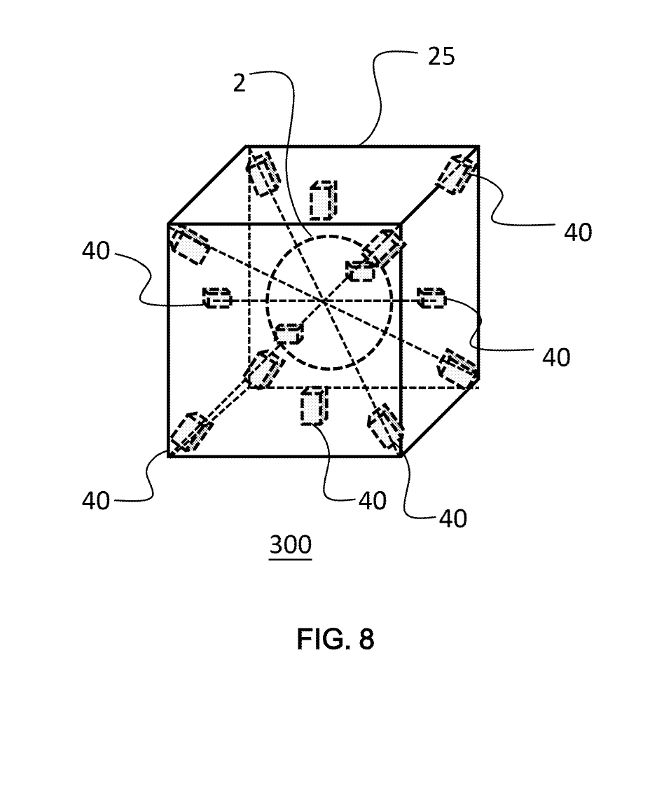

Referring to FIG. 8, in a third embodiment of the present invention, a metal meditation enhancement box 300 made of brass or copper is provided. The metal meditation enhancement box 300 includes an empty metal box 25 of about 50 mm×50 mm×50 mm in size, which essentially is a cube. A total of 14 magnets 40 made of neodymium are bonded to each of the respective corners inside the box and each of the center regions on each side (there are a total of six sides for the metal meditation box) via adhesive or epoxy glue, in which the same polarity of all of the 14 magnets 40 are pointed substantially to the center of the box.

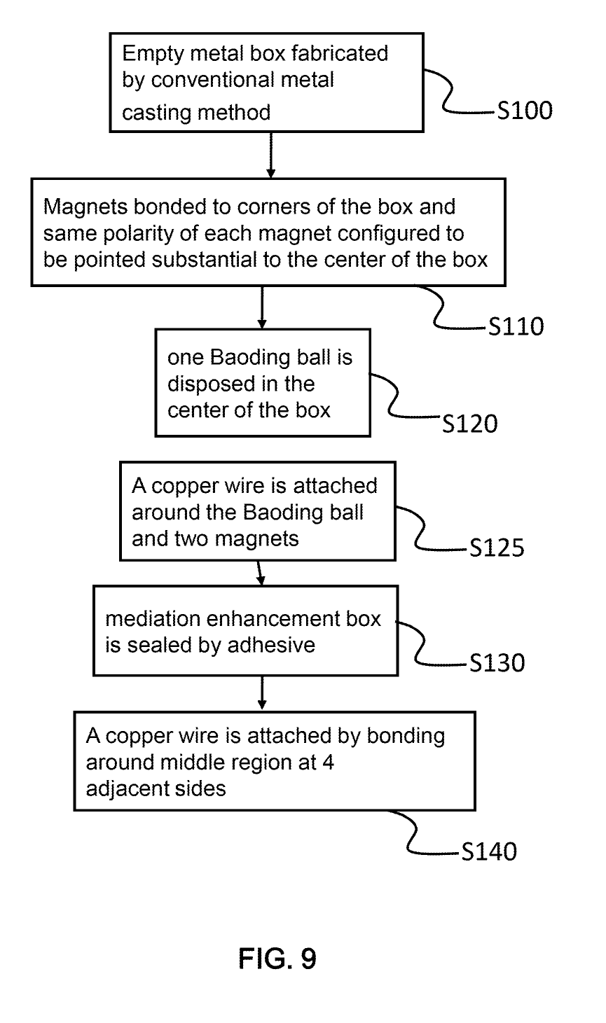

Referring to FIG. 9, a flowchart shows a manufacturing method for fabricating the metal meditation enhancement box 100 of the first embodiment of present application. In step S100, an empty metal box of about 5.08 cm×5.08 cm×5.08 cm is fabricated from copper or brass by conventional metal casting method using copper or brass, inside a set of metal molds for casting a first section, a second section, and a bottom plate. Using this metal casting method, more intricate ornamental and aesthetic designs can be configured and incorporated to the side portions of the empty metal box. In step S110, each of the 8 magnets, is bonded to one respective corner of the metal meditation enhancement box by adhesive, welding, or other conventional bonding techniques, and the same polarity of each magnet along its axial direction is configured to be pointed substantial to the center of the box. In step S120, one Baoding ball is disposed in the center of the box. In step S125, optionally, a copper wire can be bonded and conductively attached all around the Baoding ball which sits inside the meditation enhancement box, with one end of the copper wire connected to one magnet at one corner, and the other end of the copper wire connected to another magnet located in the farthest respective corner inside the metal meditation enhancement box. In step S130, the meditation enhancement box is sealed completely by adhesive, welding, or other conventional sealing techniques. In step S140, optionally, a copper wire can be attached to the mediation enhancement box by bonding the copper wire around in the middle region at 4 adjacent sides of the metal meditation enhancement box.

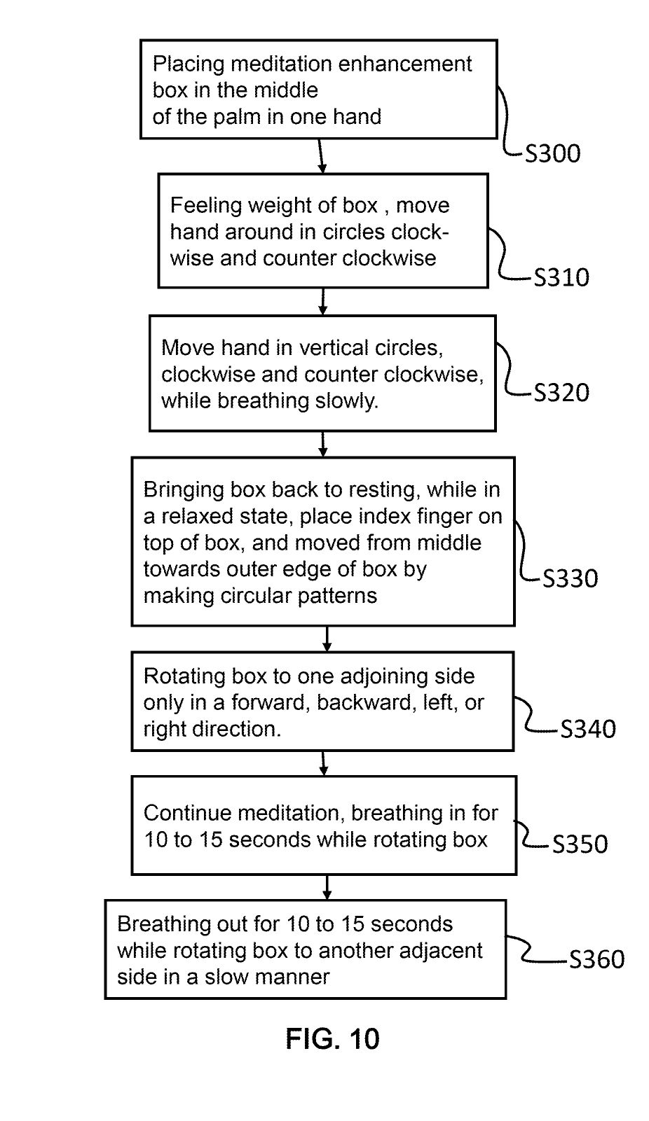

Referring to FIG. 10, an embodiment for a method for using the metal meditation enhancement box 100, 200, 300 to obtain benefits such as substantial relaxation, improved meditation quality or less amount of “mind chatter”, and stress reduction during meditation are described as follow. In step S300, a meditation participant places a metal meditation enhancement box in his palm in one hand. For a right-handed person, the left palm is laid flat for which the meditation enhancement box is to sit on, and for a left-handed person, the right palm is laid flat for which the meditation enhancement box is to sit on. In step S310, the meditation participant then takes time to feel the weight of the meditation enhancement box as it rests on his palm, and then moves his hand slowly around in circles, in a clockwise manner and then followed by moving around in circles in a counter clockwise manner. In step S320, the meditation participant then moves his hand slowly in a plurality of vertical circles, while paying close attention to how the meditation enhancement box feels in the center of his palm. As the hand movements through both the clockwise and counter clockwise circles are being performed, the meditation participant then starts to breathe slowly. In step S330, the participant then brings the box back to resting in his hand, by remaining in a resting and relaxed state as it were during the start of the exercise, and the index finger of the participant is placed on the top of the box, and the index finger is slowed moved from the middle of the box towards the outer edge of the box by making a plurality of small circular patterns. In step S340, the meditation participant holds the box in his hand, and with the other hand, slowly rotates the meditation enhancement box to one other side. At step S340, it is important that the participant only rotate the meditation enhancement box to just one adjoining side, and therefore, not rotating to multiple number of sides. Meanwhile, the participant at this step S340 has the option to rotate the box in a forward, backward, left, or right direction. In step S350, the participant breathes in for a period of 10 to 15 seconds while the meditation enhancement box is being rotated. In step S360, upon finishing of the breathing cycle, the rotating session of the meditation enhancement box by the participant should be completed. In step S370, the participant shall breathe out for a period of 10 to 15 seconds while rotating the box to another side in a slow and methodical manner.

Referring to Table 1 below, which shows the survey results in an experiment conducted with a total of 50 meditation participants who had each used one metal meditation enhancement box of instant application in one meditation session according to the method for using the meditation enhancement box described in the above steps S300 to S370, as shown in FIG. 10.

TABLE 1
Survey results taken from the experiment study conducted using
the metal meditation enhancement box in a mediation session.

| | Scoring Average | Scoring ranking | |
|---|---|---|---|
| | for all 50 | with the highest | |
| | participants (from | vote count from | |
| Test Response | 1 to 9, with 9 being | meditation | Additional |
| Category | the highest ranking) | participants | Comments |
| “I feel more | 8.0 | 8 (22 | The lowest |
| relaxed” | | participants | scoring scale |
| | | voted) | voted by |
| | | | participants |
| | | | is 5 |
| “I had less | 8.0 | 9 (26 | The lowest |
| “mind | | participants | scoring scale |
| chatter”” | | voted) | voted by |
| | | | participants |
| | | | is 5 |
| “I felt less | 8.2 | 9 (22 | The lowest |
| stressed” | | participants | scoring scale |
| | | voted) | voted by |
| | | | participants |
| | | | is 5 |

Referring to Table 1, please notice that the scoring scale or ranking is between 1 (lowest or least amount) to 9 (highest or largest amount), and the total number of meditation participants using the metal meditation enhancement box and completing the above survey is a total of 50 people. Based on the results from Table 1, the meditation enhancement box seem to be beneficial to the mediation participants for use during the meditation session so as to be able to obtain benefits or utility such as substantial relaxation, improved meditation quality or feelings of reduced “mind chatter”, and stress reduction. Indeed, the average score ranking for each test response category is about 8.0, on a scale of 1 to 9, with 9 being the highest. Meanwhile, the lowest scoring scale or ranking obtained for each test response category is at least 5 on the scale of 1 to 9, with 9 being the highest.

In the meditation enhancement box, other materials for magnets besides neodymium can also be used, such as other rare earth magnets, and other number of magnets can also be used, besides 8 or 14. Other metal fabrication method for the metal meditation enhancement box can also be used, such as, for example, metal stamping followed by bending, etc. Other bonding techniques for attaching the magnets to the inside corners of the box can be used also, such as, for example, usage of double-side adhesive tape or foam, etc. Other materials for the empty metal box besides copper or brass can also be used, such as for example, steel, copper alloy, etc.

Finally, while various embodiments have been described and illustrated, the disclosure is not to be construed as being limited thereto. Various modifications can be made to the embodiments by those skilled in the art without departing from the true spirit and scope of the disclosure as defined by the appended claims.

What is claimed is:

1. A metal meditation enhancement box, comprising:
an empty metal box made of brass or copper;
a plurality of magnets comprising of axial magnetization are bonded to each of the respective corners inside the meditation enhancement box; and
a Boading ball disposed inside the meditation enhancement box, and the Boading ball contains a chime inside to produce a chime sound during usage of the metal meditation enhancement box for meditation.

2. The metal meditation enhancement box according to claim 1, wherein the same polarity of all of the magnets are pointed substantially to the center of the meditation enhancement box.

3. The metal meditation enhancement box according to claim 1, wherein the same polarity of all of the magnets are not pointed substantially to the center of the meditation enhancement box.

4. The metal meditation enhancement box according to claim 1, wherein the empty metal box is about 5.08 cm×5.08 cm×5.08 cm in size.

5. The metal meditation enhancement box according to claim 1, wherein the empty metal box has a first section, a second section and a bottom plate, and also has a cube shape.

6. The metal meditation enhancement box according to claim 1, wherein eight magnets made of neodymium are bonded to each of the respective corners inside the meditation enhancement box via adhesive or epoxy glue.

7. The metal meditation enhancement box according to claim 1, wherein the size of each magnet is 0.635 cm×0.635 cm.

8. The metal meditation enhancement box according to claim 7, wherein the magnet has the shape of a cylinder, a cube, or a bar.

9. The metal meditation enhancement box according to claim 1, wherein the Baoding ball is about 33.5-35.5 mm in diameter, and is disposed inside the meditation enhancement box.

10. The metal meditation enhancement box according to claim 1, wherein a copper wire is further bonded and conductively attached all around the metal meditation enhancement box.

11. The metal meditation enhancement box according to claim 1, wherein a copper wire is further bonded and conductively attached all around the Baoding ball.

12. The metal meditation enhancement box according to claim 1, wherein the empty metal box is about 44 mm×44 mm×44 mm in size.

13. The metal meditation enhancement box according to claim 9, wherein 14 magnets made of neodymium are bonded to each of the respective corners inside the empty metal box and each of the center regions on each face of the empty metal box via adhesive or epoxy glue.

14. A method for using a metal meditation enhancement box in a meditation session, comprising the steps of:
placing a metal meditation enhancement box in the middle of a palm in one hand of a meditation participant;
feeling the weight of the meditation enhancement box by the meditation participant, and moving his hand slowly around clockwise and then counterclockwise in circles,
moving his hand slowly in a plurality of vertical circles, while feeling the weight of the box, initiating rhythmic breathing while moving his hand slowly around clockwise and then counterclockwise in circles;
upon reaching a preset meditation level of relaxed state, placing the index finger on the top of the box and slowing moving the index finger from the middle of the box towards the outer edge of the box by making a plurality of small circular patterns;
turning over and rotating the meditation enhancement box to one adjacent side in a forward, backward, left or right direction using a free hand of the meditation participant;
breathing in for 10 to 15 seconds while rotating the box in the same one adjacent side; and
breathing out for 10 to 15 seconds while rotating the box to another adjacent side in a forward, backward, left or right direction in a slow manner.

15. The method for using the metal meditation enhancement box as claimed in claim 14, wherein for right-handed person, the left palm is laid flat for which the meditation enhancement box is to sit on, and for left-handed person, the right palm is laid flat for which the meditation enhancement box is to sit on.
